(19) United States
(12) Patent Application Publication
(10) Pub. No.: US 2023/0149508 A1
(43) Pub. Date: May 18, 2023

(54) COMPOSITIONS AND METHODS FOR TREATING COMPLICATIONS OF VIRAL INFECTIONS AND OTHER RESPIRATORY DISORDERS

(71) Applicant: FIRSTSTRING RESEARCH, INC., Mt. Pleasant, SC (US)

(72) Inventors: Gautam GHATNEKAR, Mt. Pleasant, SC (US); Christina GREK, Mt. Pleasant, SC (US); Carissa JAMES, Mt. Pleasant, SC (US); Travis MCQUISTON, Mt. Pleasant, SC (US)

(21) Appl. No.: 17/917,673

(22) PCT Filed: Apr. 7, 2021
(86) PCT No.: PCT/US2021/026224

§ 371 Date: Oct. 7, 2022

Related U.S. Application Data
(60) Provisional application No. 63/134,462, filed on Jan. 6, 2021.
(60) Provisional application No. 63/006,498, filed on Apr. 7, 2020.

(51) Int. Cl. A61K 38/17; A61P 11/00; A61P 29/00; A61P 31/12

(52) U.S. Cl. CPC A61K 38/1709; A61K 38/17; A61P 11/00; A61P 29/00; A61P 31/12

(57) ABSTRACT

In one aspect, the present disclosure relates to treating or preventing a respiratory disease or disorder, by administering one or more compositions comprising an isolated polypeptide derived from an alpha Connexin. Exemplary respiratory diseases or disorders include acute respiratory distress syndrome (ARDS), alcoholic lung injury, acute lung injury (ALI), pulmonary fibrosis, idiopathy pulmonary fibrosis (IPF), and/or chronic obstructive pulmonary disease (COPD). In some aspects, the respiratory disease or disorder is a complication of a respiratory viral disease.

### CROSS-REFERENCE TO RELATED APPLICATIONS

This application claims priority to U.S. Provisional Application No. 63/006,498, filed on Apr. 7, 2020, and U.S. Provisional Application No. 63/134,462, filed on Jan. 6, 2021, the entire contents of each of which are hereby incorporated by reference.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: FIRS_011_01US_SeqList.txt, date recorded: Jan. 6, 2021, file size 34 kilobytes).

### BACKGROUND OF THE INVENTION

Most patients suffering from respiratory viral pneumonia will recover. However, a significant number of those affected, especially older patients and those with chronic underlying conditions, are at high risk of developing severe pneumonia and life-threatening acute respiratory distress syndrome (ARDS), a severe form of pulmonary edema that precedes respiratory failure and multiple organ dysfunction. ARDS patients require resource intensive critical care including mechanical ventilation and extended management in intensive care units. Mortality rate in ARDS patients remains ˜40% despite the availability of state-of-the-art intensive care medicine. Compounding the dismal mortality associated with ARDS, there is likely to be a significant shortage of ventilators and health care workers trained to use them in the United States during a large scale pandemic, such as what occurred with Coronavirus Disease (COVID-19) in 2019-2020.

There is a clear need in the art for an effective treatment for infection-induced lung injury that reduces ARDS incidence, need for mechanical ventilation, and deaths.

### BRIEF SUMMARY OF THE INVENTION

In one aspect, the present disclosure provides methods for treating or preventing a respiratory disease or disorder, comprising administering to the subject a composition comprising an isolated polypeptide derived from an alpha Connexin. In embodiments, the respiratory disease or disorder is associated with inflammation and/or fibrosis of the lung. In embodiments, the respiratory disease or disorder is acute respiratory distress syndrome (ARDS), alcoholic lung injury, acute lung injury (ALI), pulmonary fibrosis, idiopathy pulmonary fibrosis (IPF), and/or chronic obstructive pulmonary disease (COPD).

In embodiments, the present disclosure provides compositions and methods for treating or preventing a complication of a respiratory viral disease in a subject, comprising administering to the subject a composition comprising an isolated polypeptide derived from an alpha Connexin. Further, provided herein are compositions for use in treating or preventing a complication of a respiratory viral disease in a subject, wherein the composition comprises an isolated peptide derived from an alpha Connexin. In embodiments, the respiratory viral disease is caused by severe acute respiratory syndrome-Coronavirus 2 (SARS-CoV-2). In embodiments, the complication of the respiratory viral disease is ARDS and/or acute lung injury (ALI). For example, in embodiments, the compositions and methods provided herein are for treatment or prevention of virus (e.g., SARS-CoV-2, influenza)—induced ARDS and/or virus (e.g., SARS-CoV-2, influenza)—induced ALI and/or virus (e.g., SARS-CoV-2, influenza)—induced pulmonary fibrosis.

In embodiments, the polypeptide comprises the carboxy terminal-most 4 to 30 contiguous amino acids of the alpha Connexin. In embodiments, the alpha Connexin is Connexin 37, Connexin 40, Connexin 43, or Connexin 45. In embodiments, the polypeptide comprises an amino acid sequence selected from the group consisting of SEQ ID NO: 1, SEQ ID NO: 2, SEQ ID NO: 3, SEQ ID NO: 4, and SEQ ID NO: 5. In certain embodiments, the polypeptide comprises the amino sequence of SEQ ID NO: 2. In embodiments, the polypeptide further comprises a cellular internalization sequence. In embodiments, the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol). In embodiments, the cellular internalization sequence is Antennapedia, and wherein the sequence comprises the amino acid sequence of SEQ ID NO:7. In embodiments, the polypeptide comprises an amino acid sequence selected from the group consisting of SEQ ID NO:8, SEQ ID NO:9, SEQ ID NO:10, SEQ ID NO:11, and SEQ ID NO:12. In certain embodiments, the polypeptide comprises the amino acid sequence of SEQ ID NO:9.

In embodiments, polypeptide is administered to the subject parenterally, intranasally, intratracheally, by inhalant, or by topical intranasal administration. In embodiments, the polypeptide is administered to the subject via aerosolized delivery. In embodiments, the polypeptide is administered via an inhaler device. In embodiments, the polypeptide is administered via a dry powder inhaler, a metered dose inhaler, or a nebulizer. In embodiments, the polypeptide is administered via a ventilator. In embodiments, the polypeptide is administered to the subject in a drug loaded microcarrier formulation, such as nanoparticles or exosomes.

In embodiments, the polypeptide is administered to the subject at the onset of infection. In embodiments, the polypeptide is administered to the subject after infection with a virus that causes the respiratory viral disease and prior to the onset of symptoms of the respiratory viral disease. Accordingly, in embodiments, the polypeptide is administered after the subject is identified as having been infected with the virus (e.g., SARS-CoV-2) or after the subject has been identified as being suspected of being infected with the virus, or as being at risk of infection with the virus. In embodiments, the polypeptide is administered prior to infection with the virus. In embodiments, the polypeptide is administered prior to the onset of symptoms of the respiratory viral disease. In embodiments, the polypeptide is administered after the onset of symptoms of the respiratory viral disease. In embodiments, the compositions and methods provided herein prevent the onset of, or mitigate the progression of, or reverse progression of, the respiratory viral disease. In embodiments, the compositions and methods provided herein maintain lung function after the onset of the respiratory viral disease.

In embodiments, the compositions and methods provided herein treat or prevent lung injury and/or respiratory disorders. Exemplary indications in acute and chronic lung injuries and/or respiratory disorders include ARDS, alcoholic lung injury, ALI, pulmonary fibrosis, idiopathy pulmonary fibrosis (IPF), and chronic obstructive pulmonary disease (COPD). Thus, in embodiments, the compositions and methods provided herein treat or prevent ARDS, alcoholic lung syndrome, and/or ALI. In embodiments, the compositions and methods provided herein treat or prevent SARS-CoV-2 related ARDS, ALI, and/or pulmonary fibrosis.

### BRIEF DESCRIPTION OF THE DRAWINGS

FIG. 1 provides a schematic view of the aCT1 peptide.

FIG. 2. shows that intranasal pre- and post-treatment with aCT1 provides significant protection against LPS-induced mortality. C57BL/6 mice were pretreated with either 1000 uM aCT1, 1000 uM scrambled aCT1, or vehicle control prior to inoculation with a lethal dose (35 mg/kg/mouse) of LPS. A further group was treated with aCT1 6 hrs post inoculation of LPS. n=10/group (p<0.01 LPS vs LPS pre-aCT1, p<0.01 LPS vs LPS post aCT1).

FIG. 3 shows that aCT1 prolongs survival in a mouse model of sepsis. Mice were administered vehicle control, control peptide, or 1, 10, 100, 1000 μM of aCT1 6 hr after cecal-ligation and puncture (CLP) procedure. Treatment of mice with 1000 μM of aCT1 significantly improved survival p<0.001, n=10 in all groups.

FIG. 4 shows that aCT1 reduces intra-alveolar neutrophil accumulation. Histopathological analysis using H&E staining of lungs at 12 hrs post CLP injury shows delivery of 1000 uM aCT1 significantly decreased immune cell infiltration and alveolar edema. Representative images are shown.

FIG. 5 shows that normal lung function is not impaired by aerosolized aCT1 treatment in healthy animals. Delivery of 5 mg/kg aCT1 was not associated with alterations in lung function or overall respiratory health in healthy mice, as measured by Penh (enhanced pause).

FIG. 6A provides immunostaining images demonstrating that aCT1 localized to epithelial cells and endothelial cells in the conducting airways and alveoli. FIG. 6B shows quantification of aCT1 staining in bronchial and alveolar epithelial cells.

FIG. 7 shows that pre-treatment of normal human bronchial epithelial cells (NHBE) with aCT1 prevents oxidative stress induced pulmonary injury. Pre-treatment significantly increased TER readings after exposure to 100 mM H₂0₂. (n=3, compared to H₂O₂ *:p<0.05, **:p<0.01, ***:p<0.0001)

FIG. 8 shows that aCT1 provides protection from LPS induced injury. Human lung microvascular endothelial cells (HLMEC) were exposed to 10 μg LPS. Cells were treated with aCT1 prior to exposure or 24 hrs post LPS insult, scrambled control, or sham control. Pre-treatment and treatment at 24 hrs significantly improved endothelial TER readings. (n=3, compared to LPS ***:p<0.0001).

### DETAILED DESCRIPTION

Provided herein are methods for treating or preventing diseases and disorders that are complications of viral respiratory infections, such as infection with SARS-CoV-2 (virus causing COVID-19). In embodiments, the respiratory viral infection is selected from Severe Acute Respiratory Syndrome-Corona Virus (SARS-CoV), Middle East Respiratory Syndrome virus (MERS-CoV), human HCoV-229E, HCoV-OC43, HCoV-NL63 and HCoV-HKU1. In embodiments, the virus infection is a coronavirus infection. In embodiments, the coronavirus is an alpha coronavirus (e.g., HCoV-EE29, HCoV-NL63) or a beta coronavirus (e.g., HCoV-OC43, HCoV-HKU1, MERS-CoV, or SARS-CoV). In embodiments, the coronavirus infection is SARS-CoV (e.g. SARS-CoV-1, SARS-CoV-2). In certain embodiments, the SARS-CoV is SARS-CoV-2 (virus causing COVID-19). In embodiments, the respiratory viral infection is an influenza viral infection or a parainfluenza virus infection (PIV) infection. In embodiments, the influenza viral infection is selected from the group consisting of Influenza A, Influenza B, and Influenza C viral infections. In some embodiments, the Influenza A virus comprises H1N1, H2N2, H3N2, H5N1, H7N7, H1N2, H9N2, H7N2, H7N3, or H10N7 subtypes. In embodiments, the respiratory viral infection is respiratory syncytial virus (RSV).

The highly pathogenic SARS-CoV-2 is associated with rapid virus replication, massive inflammatory cell infiltration and elevated pro-inflammatory cytokine/chemokine responses. Infection and the ensuing inflammatory response can result in acute lung injury (ALI) and lead to acute respiratory distress syndrome (ARDS), pulmonary fibrosis, and death. No specific treatment exists except for supportive care including mechanical ventilation, which can itself further exacerbate respiratory distress. Mortality rates in patients requiring mechanical ventilation are high.

Interconnected epithelial cells line the pulmonary air spaces, forming a physiological barrier separating inspired air from fluid-filled tissues and providing a surface for gas exchange. Integrity of this barrier is essential for pulmonary function; where disruption results in the accumulation of fluid in the alveoli and respiratory failure². SARS-CoV-2, similar to the previous coronaviruses MERS-CoV and SARS-CoV, targets epithelial cells lining the airways for viral entry and replication^{3,4}. The virus causes severe lesions and shedding of the bronchial and alveolar epithelial cells lining airways⁵. The resulting diffuse alveolar damage primes the lung for edema and fibrosis. In many patients infected with SARS-CoV-2, particularly those in high risk groups, this progresses to a severe pulmonary pneumonia and acute respiratory distress as the lung becomes fluid filled and fibrotic⁶. Excessive inflammation elicited in response to viral infection and lung injury exacerbates the severity of COVID-19 as proinflammatory cytokines and immune cell infiltration exacerbate lung fibrosis and thickening of the airway walls, further compromising the lung's ability to permit gas exchange⁷.

Severe pneumonia resulting from loss of pulmonary epithelial barrier function and a faulty immune response results in disease progression to acute respiratory distress syndrome in many patients with respiratory viral infections, such as those caused by SARS Coronaviruses or influenza. Without wishing to be bound by theory, the alpha Connexin peptides provided herein provide a therapeutic intervention that preserves epithelial integrity and dampens inflammation in infected lungs. Accordingly, the methods provided herein include mitigation of development of acute respiratory failure in hospitalized patients (e.g., patients having respiratory viral infections, such as those caused by SARS coronaviruses or influenza), which lessens the need for intensive mechanical ventilation and dramatically improves patient survival.

Without wishing to be bound by theory, alpha Connexin peptides provided herein directly target and repair the damaged cells lining airspaces and vasculature in the virus infected lung, thus restoring barrier integrity and directly addressing the cause of ARDS. While antiviral therapies in development for respiratory viral infections can limit viral replication and accelerate viral clearance, these therapies will not address the need for rapid repair of the pulmonary epithelium in critically ill patients to prevent progression of severe pneumonia to ARDS. In embodiments, therapeutically targeting intercellular junctions in the infected lung via the alpha Connexin peptide (e.g., aCT1) stabilizes epithelial barriers, mitigates the pathological immune response, and prevents development of acute respiratory failure to improve survival and decrease need for ventilation.

Accordingly, in embodiments, the present disclosure provides methods for treating a respiratory disease, disorder, or condition by administering to a subject in need thereof a polypeptide provided herein (e.g., an alpha Connexin polypeptide, e.g., aCT polypeptide). In embodiments, the respiratory disease, disorder or condition is COVID-19 (coronavirus disease) mediated acute respiratory distress syndrome (ARDS). In embodiments, the respiratory disease, disorder, or condition is ARDS which is not associated with a viral infection, or which is not triggered by a viral infection. In embodiments, the respiratory disease, disorder, or condition is ARDS that is triggered by a secondary insult that may or may not be a viral infection. For example, in embodiments, the respiratory disease, disorder, or condition may be an underlying condition or may be associated with an underlying condition, wherein a secondary insult (e.g., a lung injury and/or viral respiratory infection) may result in ARDS. In embodiments, the respiratory disease or condition is alcoholic lung injury or alcoholic lung syndrome. In embodiments, the respiratory disease or condition is pulmonary fibrosis, IPF, or COPD. The compositions and methods provided herein are useful for treatment of chronic lung injuries and/or respiratory disorders (e.g., pulmonary fibrosis, IPF, COPD, ARDS, and/or ALI) whether or not associated with or triggered by a viral infection. In embodiments, the present disclosure provides compositions and methods for treating or preventing a respiratory disease, disorder, or condition, comprising administering a composition provided herein directly into the lungs via nebulization.

Impaired wound healing and a dysfunctional immune response render the alcoholic lung unable to repair its chronically damaged epithelium. The cumulative effect of chronic alcohol ingestion on pulmonary gap and tight junctions causes a loss of barrier integrity that defines alcoholic lung syndrome, priming the lung for acute lung injury and ARDS. Alcoholic lung syndrome often remains undiagnosed until hospitalization due to a secondary insult, as compensatory upregulation of fluid transport in the alcoholic lung allows the syndrome to remain subclinical. When faced with an acute secondary insult, these compensatory mechanisms are quickly overwhelmed, and the alcohol injured lung develops an exaggerated and lethal response to insult that precipitates respiratory failure. 25% of patients have alcohol use disorder at the time of hospital admission in the United States. In-hospital mortality for alcoholics is double that of matched non-alcoholic patients, and this is primarily attributed to the increased risk of developing ARDS. In embodiments, the present disclosure provides methods for treating and/or preventing development of ARDS comprising administration of a composition provided herein (e.g., aCT1) to a patient upon hospital or ICU admission. In embodiments, the patient is an alcoholic patient. Without wishing to be bound by theory, in embodiments, compositions provided herein (e.g., aCT1) restore the pulmonary barrier in a patient having alcoholic lung injury, and protect the lung from development of ARDS.

The polypeptides provided herein comprise a carboxy-terminal amino acid sequence of an alpha Connexin, or a conservative variant thereof. In embodiments, the polypeptide comprises or consists of the amino acid sequence RPRPDDLEI (SEQ ID NO: 2). In embodiments, the polypeptide is aCT1, as described herein. The term “aCT1” is used interchangeably herein with “aCT1” and “ACT1”. aCT1 is a 25 aa peptide having the amino acid sequence RQPKIWFPNRRKPWKKRPRPDDLEI (SEQ ID NO: 9). In embodiments, the compositions and methods provided herein are related to preventing, treating, and/or mitigating the progression of complications from viral infections. In embodiments, the compositions and methods provided herein are related to preventing, treating, and/or mitigating the progression of respiratory and pulmonary complications of viral infections, such as acute respiratory distress syndrome (ARDS) or acute lung injury (ALI), by administering aCT1 to a subject in need thereof. For example, in embodiments, the compositions and methods herein are related to preventing, treating, and/or mitigating the progression of ARDS and/or ALI in patients suffering from a respiratory infection such as a SARS-CoV-2 infection, by administering aCT1 to a subject in need thereof. In embodiments, the aCT1 polypeptide provided herein is for use in preventing, treating, and/or mitigating the progression of respiratory and pulmonary complications of viral infections, such as ARDS or ALI. In embodiments, provided herein are uses of aCT1 in the manufacture of a medicament for preventing or treating respiratory and pulmonary complications of viral infections, such as ARDS, ALI, and/or fibrosis.

The herein provided polypeptide can be any polypeptide comprising the carboxy-terminal most amino acids of an alpha Connexin, wherein the polypeptide does not comprise the full-length alpha Connexin protein. Thus, in embodiments, the provided polypeptide does not comprise the cytoplasmic N-terminal domain of the alpha Connexin. In embodiments, the provided polypeptide does not comprise the two extracellular domains of the alpha Connexin. In embodiments, the provided polypeptide does not comprise the four transmembrane domains of the alpha Connexin. In embodiments, the provided polypeptide does not comprise the cytoplasmic loop domain of the alpha Connexin. In embodiments, the provided polypeptide does not comprise that part of the sequence of the cytoplasmic carboxyl terminal domain of the alpha Connexin proximal to the fourth transmembrane domain. There is a conserved proline or glycine residue in alpha Connexins consistently positioned some 17 to 30 amino acids from the carboxyl terminal-most amino acid For example, for human Cx43 a proline residue at amino acid 363 is positioned 19 amino acids back from the carboxyl terminal most isoleucine. In another example, for chick Cx43 a proline residue at amino acid 362 is positioned 18 amino acids back from the carboxyl terminal-most isoleucine. In another example, for human Cx45 a glycine residue at amino acid 377 is positioned 19 amino acids back from the carboxyl terminal most isoleucine. In another example for rat Cx33, a proline residue at amino acid 258 is positioned 28 amino acids back from the carboxyl terminal most methionine. Thus, in embodiments, the provided polypeptide does not comprise amino acids proximal to said conserved proline or glycine residue of the alpha Connexin. Thus, the provided polypeptide can comprise the c-terminal-most 4 to 30 amino acids of the alpha Connexin, including the c-terminal most 4, 5, 6, 7, 8, 9, 10, 11, 12, 13, 14, 15, 16, 17, 18, 19, 20, 21, 22, 23, 24, 25, 26, 27, 28, 29, 30 amino acids of the alpha Connexin. Exemplary alpha Connexin polypeptides are disclosed in U.S. Pat. Nos. 7,786,074; 7,888,319; 8,357,668; 8,809,257; 8,916,515; 8,859,733; 8,846,605; 9,161,984; 9,394,351; 9,408,381; 9,844,214; 9,855,313; 10,398,140; and 10,398,757, and/or International Patent Application No. PCT/US2018/000035, the entire contents of each of which are hereby incorporated by reference.

Connexins are the sub-unit protein of the gap junction channel, which is responsible for intercellular communication (Goodenough and Paul, 2003). Based on patterns of conservation of nucleotide sequence, the genes encoding Connexin proteins are divided into two families termed the alpha and beta Connexin genes. The carboxy-terminal-most amino acid sequences of alpha Connexins are characterized by multiple distinctive and conserved features. This conservation of organization is consistent with the ability of ACT peptides to form distinctive 3D structures, interact with multiple partnering proteins, mediate interactions with lipids and membranes, interact with nucleic acids including DNA, transit and/or block membrane channels and provide consensus motifs for proteolytic cleavage, protein cross-linking, ADP-ribosylation, glycosylation and phosphorylation. Thus, the provided polypeptide interacts with a domain of a protein that normally mediates the binding of said protein to the carboxy-terminus of an alpha Connexin. For example, nephroblastoma overexpressed protein (NOV) interacts with a Cx43 c-terminal domain (Fu et al., J Biol. Chem. 2004 279(35):36943-50). It is considered that this and other proteins interact with the carboxy-terminus of alpha Connexins and further interact with other proteins forming a macromolecular complex. Thus, the provided polypeptide can inhibit the operation of a molecular machine, such as, for example, one involved in regulating the aggregation of Cx43 gap junction channels.

The ACT sequence of the provided polypeptide can be from any alpha Connexin. Thus, the alpha Connexin component of the provided polypeptide can be from a human, murine, bovine, monotrene, marsupial, primate, rodent, cetacean, mammalian, avian, reptilian, amphibian, piscine, chordate, protochordate or other alpha Connexin. Thus, the provided polypeptide can comprise an ACT of a Connexin selected from the group consisting of mouse Connexin 47, human Connexin 47, Human Connexin 46.6, Cow Connexin 46.6, Mouse Connexin 30.2, Rat Connexin 30.2, Human Connexin 31.9, Dog Connexin 31.9, Sheep Connexin 44, Cow Connexin 44, Rat Connexin 33, Mouse Connexin 33, Human Connexin 36, mouse Connexin 36, rat Connexin 36, dog Connexin 36, chick Connexin 36, zebrafish Connexin 36, morone Connexin 35, morone Connexin 35, Cynops Connexin 35, Tetraodon Connexin 36, human Connexin 37, chimp Connexin 37, dog Connexin 37, Cricetulus Connexin 37, Mouse Connexin 37, Mesocricetus Connexin 37, Rat Connexin 37, mouse Connexin 39, rat Connexin 39, human Connexin 40.1, Xenopus Connexin 38, Zebrafish Connexin 39.9, Human Connexin 40, Chimp Connexin 40, dog Connexin 40, cow Connexin 40, mouse Connexin 40, rat Connexin 40, Cricetulus Connexin 40, Chick Connexin 40, human Connexin 43, Cercopithecus Connexin 43, Oryctolagus Connexin 43, Spermophilus Connexin 43, Cricetulus Connexin 43, Phodopus Connexin 43, Rat Connexin 43, Sus Connexin 43, Mesocricetus Connexin 43, Mouse Connexin 43, Cavia Connexin 43, Cow Connexin 43, Erinaceus Connexin 43, Chick Connexin 43, Xenopus Connexin 43, Oryctolagus Connexin 43, Cyprinus Connexin 43, Zebrafish Connexin 43, Danio aequipinnatus Connexin 43, Zebrafish Connexin 43.4, Zebrafish Connexin 44.2, Zebrafish Connexin 44.1, human Connexin 45, chimp Connexin 45, dog Connexin 45, mouse Connexin 45, cow Connexin 45, rat Connexin 45, chick Connexin 45, Tetraodon Connexin 45, chick Connexin 45, human Connexin 46, chimp Connexin 46, mouse Connexin 46, dog Connexin 46, rat Connexin 46, Mesocricetus Connexin 46, Cricetulus Connexin 46, Chick Connexin 56, Zebrafish Connexin 39.9 cow Connexin 49, human Connexin 50, chimp Connexin 50, rat Connexin 50, mouse Connexin 50, dog Connexin 50, sheep Connexin 49, Mesocricetus Connexin 50, Cricetulus Connexin 50, Chick Connexin 50, human Connexin 59, or other alpha Connexin.

The 20-30 carboxy-terminal-most amino acid sequence of alpha Connexins are characterized by a distinctive and conserved organization. This distinctive and conserved organization includes a type II PDZ binding motif (Φx-Φ); wherein x=any amino acid and Φ=a Hydrophobic amino acid; e.g., Table 2, BOLD) and proximal to this motif, Proline (P) and/or Glycine (G) hinge residues; a high frequency phospho-Serine (S) and/or phospho-Threonine (T) residues; and a high frequency of positively charged Arginine (R), Lysine (K) and negatively charged Aspartic acid (D) or Glutamic acid (E) amino acids. For many alpha Connexins, the P and G residues occur in clustered motifs proximal to the carboxy-terminal type II PDZ binding motif. The S and T phosphor-amino acids of most alpha Connexins also are typically organized in clustered, repeat-like motifs. This organization is particularly the case for Cx43, where 90% of 20 carboxyl terminal-most amino acids are comprised of the latter seven amino acids. In a further example of the high conservation of the sequence, ACT peptide organization of Cx43 is highly conserved from humans to fish.

Thus, in one aspect, the provided polypeptide comprises one, two, three or all of the amino acid motifs selected from the group consisting of 1) a type II PDZ binding motif, 2) Proline (P) and/or Glycine (G) hinge residues; 3) clusters of phospho-Serine (S) and/or phospho-Threonine (T) residues; and 4) a high frequency of positively charged Arginine (R) and Lysine (K) and negatively charged Aspartic acid (D) and/or Glutamic acid (E) amino acids). In another aspect, the provided polypeptide comprises a type II PDZ binding motif at the carboxy-terminus, Proline (P) and/or Glycine (G) hinge residues proximal to the PDZ binding motif, and positively charged residues (K, R, D, E) proximal to the hinge residues.

PDZ domains were originally identified as conserved sequence elements within the postsynaptic density protein PSD95/SAP90, the Drosophila tumor suppressor dlg-A, and the tight junction protein ZO-1. Although originally referred to as GLGF or DHR motifs, they are now known by an acronym representing these first three PDZ-containing proteins (PSD95/DLG/ZO-1). These 80-90 amino acid sequences have now been identified in well over 75 proteins and are characteristically expressed in multiple copies within a single protein. Thus, in one aspect, the provided polypeptide can inhibit the binding of an alpha Connexin to a protein comprising a PDZ domain. The PDZ domain is a specific type of protein-interaction module that has a structurally well-defined interaction ‘pocket’ that can be filled by a PDZ-binding motif, referred to herein as a “PDZ motif”. PDZ motifs are consensus sequences that are normally, but not always, located at the extreme intracellular carboxyl terminus. Four types of PDZ motifs have been classified: type I (S/T-x-Φ), type II (Φ-x-Φ), type III (Ψ-x-Φ) and type IV (D-x-V), where x is any amino acid, Φ is a hydrophobic residue (V, I, L, A, G, W, C, M, F) and Ψ is a basic, hydrophilic residue (H, R, K). (Songyang, Z., et al. 1997. Science 275, 73-77). Thus, in one aspect, the provided polypeptide comprises a type II PDZ binding motif

In embodiments, the provided polypeptide comprises the c-terminal sequence of human Cx43. Thus, in embodiments, the polypeptide comprises or consists of the amino acid sequence SEQ ID NO:1 (PSSRASSRPRPDDLEI) or SEQ ID NO:2 (RPRPDDLEI).

When specific proteins are referred to herein, variants, derivatives, and fragments are contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known and include, for example, M13 primer mutagenesis and PCR mutagenesis. Amino acid substitutions are typically of single residues, but can occur at a number of different locations at once; insertions usually will be on the order of about from 1 to 10 amino acid residues. Substitutions, deletions, insertions or any combination thereof may be combined to arrive at a final construct. Substitutional variants are those in which at least one residue has been removed and a different residue inserted in its place.

For example, the replacement of one amino acid residue with another that is biologically and/or chemically similar is known to those skilled in the art as a conservative substitution. For example, a conservative substitution would be replacing one hydrophobic residue for another, or one polar residue for another. Conservatively substituted variations of each explicitly disclosed sequence are included within the polypeptides provided herein.

Typically, conservative substitutions have little to no impact on the biological activity of a resulting polypeptide. In a particular example, a conservative substitution is an amino acid substitution in a peptide that does not substantially affect the biological function of the peptide. A peptide can include one or more amino acid substitutions, for example 2-10 conservative substitutions, 2-5 conservative substitutions, 4-9 conservative substitutions, such as 2, 5 or 10 conservative substitutions.

A polypeptide can be produced to contain one or more conservative substitutions by manipulating the nucleotide sequence that encodes that polypeptide using, for example, standard procedures such as site-directed mutagenesis or PCR. Alternatively, a polypeptide can be produced to contain one or more conservative substitutions by using standard peptide synthesis methods. An alanine scan can be used to identify which amino acid residues in a protein can tolerate an amino acid substitution. In one example, the biological activity of the protein is not decreased by more than 25%, for example not more than 20%, for example not more than 10%, when an alanine, or other conservative amino acid (such as those listed below), is substituted for one or more native amino acids.

It is understood that there are numerous amino acid and peptide analogs which can be incorporated into the disclosed compositions. For example, there are numerous D amino acids. The opposite stereoisomers of naturally occurring peptides are disclosed, as well as the stereoisomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994), all of which are herein incorporated by reference at least for material related to amino acid analogs).

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

Thus, the provided polypeptide can comprise a conservative variant of the c-terminus of an alpha Connexin (ACT). It is understood that one way to define any variants, modifications, or derivatives of the disclosed genes and proteins herein is through defining the variants, modification, and derivatives in terms of sequence identity (also referred to herein as homology) to specific known sequences. Specifically disclosed are variants of the nucleic acids and polypeptides herein disclosed which have at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the stated or known sequence. Those of skill in the art readily understand how to determine the sequence identity of two proteins or nucleic acids. For example, the sequence identity can be calculated after aligning the two sequences so that the sequence identity is at its highest level. Another way of calculating sequence identity can be performed by published algorithms.

Thus, the provided polypeptide can comprise an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to the c-terminus of an alpha Connexin (ACT). Thus, in one aspect, the provided polypeptide comprises an amino acid sequence with at least 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent sequence identity to SEQ ID NO:1, SEQ ID NO: 2, or any sequence provided herein.

In embodiments, the polypeptide comprises a cellular internalization transporter or sequence. The cellular internalization sequence can be any internalization sequence known or newly discovered in the art, or conservative variants thereof. Non-limiting examples of cellular internalization transporters and sequences include Antennapedia sequences, TAT, HIV-Tat, Penetratin, Antp-3A (Antp mutant), Buforin II, Transportan, MAP (model amphipathic peptide), K-FGF, Ku70, Prion, pVEC, Pep-1, SynB1, Pep-7, HN-1, BGSC (Bis-Guanidinium-Spermidine-Cholesterol, and BGTC (Bis-Guanidinium-Tren-Cholesterol). Exemplary cell internalization transporters are provided in Table 1.

TABLE 1
Exemplary cell internalization sequences

| | | SEQ ID |
|---|---|---|
| Name | Sequence | NO |
| Antp | RQPKIWFPNRRKPWKK | (SEQ ID |
| | | NO: 7) |
| HIV-Tat | GRKKRRQRPPQ | (SEQ ID |
| | | NO: 14) |
| Penetratin | RQIKIWFQNRRMKWKK | (SEQ ID |
| | | NO: 15) |
| Antp-3A | RQIAIWFQNRRMKWAA | (SEQ ID |
| | | NO: 16) |
| Tat | RKKRRQRRR | (SEQ ID |
| | | NO: 17) |
| Buforin II | TRSSRAGLQFPVGRVH | (SEQ ID |
| | RLLRK | NO: 18) |
| Transportan | GWTLNSAGYLLGKINKAL | (SEQ ID |
| | AALAKKIL | NO: 19) |
| model | KLALKLALKALKAALKLA | (SEQ ID |
| amphipathic | | NO: 20) |
| peptide | | |
| (MAP) | | |
| K-FGF | AAVALLPAVLLALLAP | (SEQ ID |
| | | NO: 21) |
| Ku70 | VPMLK-PMLKE | (SEQ ID |
| | | NO: 22) |
| Prion | MANLGYWLLALFVT | (SEQ ID |
| | MWTDVGLCKKRPKP | NO: 23) |
| pVEC | LLIILRRRIRKQAHAHSK | (SEQ ID |
| | | NO: 24) |
| Pep-1 | KETWWETWWTEWSQP | (SEQ ID |
| | KKKRKV | NO: 25) |
| SynBI | RGGRLSYSRRRFSTSTGR | (SEQ ID |
| | | NO: 26) |
| Pep-7 | SDLWEMMMVSLACQY | (SEQ ID |
| | | NO: 27) |
| HN-1 | TSPLNIHNGQKL | (SEQ ID |
| | | NO: 28) |
| BGSC (Bis- | | |
| Guanidinium- | | |
| Spermidine- | | |
| Cholesterol) | | |
| BGTC (Bis- | | |
| Guanidinium- | | |
| Tren- | | |
| Cholesterol) | | |

Any other internalization sequences now known or later identified can be combined with a peptide of the invention.

The provided polypeptide can comprise any ACT sequence (e.g, any of the ACT peptides disclosed herein) in combination with any of the herein provided cell internalization sequences. Examples of said combinations are provided in Table 2. Thus, the provided polypeptide can comprise an Antennapedia sequence comprising amino acid sequence SEQ ID NO:7. Thus, the provided polypeptide can comprise the amino acid sequence SEQ ID NO:8, SEQ ID NO:9, SEQ ID NO:10, SEQ ID NO:11, or SEQ ID NO: 12.

TABLE 2
ACT Polypeptides with Cell Internalization
Sequences (CIS) ACT Polypeptides
with Cell Internalization Sequences (CIS)

| CIS/ACT | Sequence | SEQ ID NO |
|---|---|---|
| Antp/ | RQPKIWFPNRRKPWKK | SEQ ID NO: 8 |
| ACT 2 | PSSRASSRASSRPRPDDLEI | |
| Antp/ | RQPKIWFPNRRKPWKK RPRP | SEQ ID NO: 9 |
| ACT 1 | DDLEI | |
| Antp/ | RQPKIWFPNRRKPWKK RPRP | SEQ ID NO: 10 |
| ACT 3 | DDLEV | |
| Antp/ | RQPKIWFPNRRKPWKK RPRP | SEQ ID NO: 11 |
| ACT 4 | DDVPV | |
| Antp/ | RQPKIWFPNRRKPWKK KARS | SEQ ID NO: 12 |
| ACT 5 | DDLSV | |
| HIV-Tat/ | GRKKRRQRPPQ RPRPDDLEI | SEQ ID NO: 56 |
| ACT 1 | | |
| Penetratin | RQIKIWFQNRRMKWKK RPRP | SEQ ID NO: 57 |
| /ACT 1 | DDLEI | |
| Antp-3A/ | RQIAIWFQNRRMKWAA RPRP | SEQ ID NO: 58 |
| ACT 1 | DDLEI | |
| Tat/ | RKKRRQRRR RPRPDDLEI | SEQ ID NO: 59 |
| ACT 1 | | |
| Buforin | TRSSRAGLQFPVGRVHRLLRK | SEQ ID NO: 60 |
| II/ | RPRPDDLEI | |
| ACT 1 | | |
| Transport | GWTLNSAGYLLGKINKALAAL | SEQ ID NO: 61 |
| an/ | AKKILRPRPDDLEI | |
| ACT 1 | | |
| MAP/ | KLALKLALKALKAALKLA RPR | SEQ ID NO: 62 |
| ACT 1 | PDDLEI | |
| K-FGF/ | AAVALLPAVLLALLAP RPRPD | SEQ ID NO: 63 |
| ACT 1 | DLEI | |
| Ku70/ | VPMLKPMLKE RPRPDDLEI | SEQ ID NO: 64 |
| ACT 1 | | |
| Prion/ | MANLGYWLLALFVTMWTDVGLC | SEQ ID NO: 65 |
| ACT 1 | KKRPKPRPRPDDLEI | |
| pVEC/ | LLIILRRRIRKQAHAHSK RPR | SEQ ID NO: 66 |
| ACT 1 | PDDLEI | |
| Pep-1/ | KETWWETWWTEWSQPKKKRKV | SEQ ID NO: 67 |
| ACT 1 | RPRPDDLEI | |
| SynB1/ | RGGRLSYSRRRFSTSTGR RPRP | SEQ D NO: 68 |
| ACT 1 | DDLEI | |
| Pep-7/ | SDLWEMMMVSLACQY RPR | SEQ ID NO: 69 |
| ACT 1 | PDDLEI | |
| HN-1/ | TSPLNIHNGQKL RPRPDDLEI | SEQ ID NO: 70 |
| ACT 1 | | |

Also provided are isolated nucleic acids encoding the polypeptides provided herein. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Thus, provided is an isolated nucleic acid encoding a polypeptide comprising the amino acid sequence SEQ ID NO:1, SEQ ID NO:2, SEQ ID NO:3, SEQ ID NO:4, SEQ ID NO:5, SEQ ID NO:7, SEQ ID NO:8, SEQ ID NO:9, SEQ ID NO:10, SEQ ID NO:11, or SEQ ID NO:12.

In embodiments, provided herein is a composition comprising one or more of the herein provided polypeptides, nucleic acids, or vectors in a pharmaceutically acceptable carrier. For example, provided is a composition comprising SEQ ID NO:2 or SEQ ID NO:9 in a pharmaceutically acceptable carrier. In embodiments, the composition comprises one or more of the herein provided polypeptides encapsulated in a microcarrier. For example, in embodiments, the composition comprises one or more of the herein provided polypeptides, wherein the polypeptides are in a nanoparticle or exosome.

By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered parenterally, intranasally, intratracheally, by inhalant, or by topical intranasal administration. As used herein, “topical intranasal administration” means delivery of the compositions into the nose and nasal passages through one or both of the nares and can comprise delivery by a spraying mechanism or droplet mechanism, or through aerosolization. Administration of the compositions by inhalant can be through the nose or mouth via delivery by a spraying or droplet mechanism. Intratracheal administration may include intratracheal injection, instillation, or inhalation. Delivery can also be directly to any area of the respiratory system (e.g., lungs) via intubation, or via a ventilator. Delivery may be via a dry powder inhaler, a metered dose inhaler, a nebulizer (e.g., atomizer jet nebulizer or ultrasonic nebulizer), through a mechanical ventilator, or any other means of intranasal, inhalant, intratracheal, or pulmonary administration. Delivery via any of the above administration routes may be in the form of a drug loaded microcarrier formulation, such as nanoparticles or exosomes.

In embodiments, the compositions provided herein comprise drug loaded microcarrier formulations comprising nanoparticles or exosomes. In embodiments, the size of the nanoparticles is from about 100 nm to about 1000 nm, or about 100 nm to about 500 nm, or about 200 nm to about 250 nm, or about 100 nm to about 200 nm.

In embodiments, the composition is administered to the subject at a dose of from about 0.1 mg/kg to about 50 mg/kg. In embodiments, the composition is administered to the subject at a dose of about 0.1 mg/kg, about 0.5 mg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, or about 50 mg/kg. In embodiments, the composition is administered in a daily dosing regimen. In embodiments, the composition is administered to the subject in a formulation comprising about 1μM to about 10,000 μM of the polypeptide, or about 10 μM to about 9,000 μM, or about 50 μM to about 5,000 μM, or about 100 μM to about 2,000 μM, or about 200 μM to about 2,000 μM, or about 200 μM to about 1,000 μM, or about 50 μM to about 1,500 μM of the polypeptide, or about 100 μM to about 1,000 μM of the polypeptide, or about 500 to about 1,500 μM of the polypeptide. In embodiments, the composition is administered to the subject in a formulation comprising about 1 μM , about 5 μM, about 50 μM, about 100 μM, about 150 μM, about 200 μM, about 300 μM, about 400 μM, about 500 μM, about 600 μM, about 700 μM, about 800 μM, about 900 μM, about 1,000 μM, about 1,500 μM, about 2,000 μM, about 3,000 μM, about 4,000 μM, about 5,000 μM, about 6,000 μM, about 7,000 μM, about 8,000 μM, about 9,000 μM, or about 10,000 μM of the polypeptide.

As used herein, “subject” include vertebrates, more specifically a mammal (e.g., a human, horse, pig, rabbit, dog, sheep, goat, non-human primate, cow, cat, guinea pig or rodent), a fish, a bird or a reptile or an amphibian. In embodiments, the subject is a human subject. The term does not denote a particular age or sex. Thus, adult and newborn subjects, as well as fetuses, whether male or female, are intended to be covered. In embodiments, a patient refers to a subject afflicted with a disease or disorder. In embodiments, a patient population refers to a particular, defined set of subjects having a disease or disorder or at risk of developing a particular disease or disorder.

As used herein, “inhibit,” “inhibiting,” and “inhibition” mean to decrease an activity, response, condition, disease, or other biological parameter. This can include, but is not limited to, the complete loss of activity, response, condition, or disease. Thus, the reduction can be a 10, 20, 30, 40, 50, 60, 70, 80, 90, 100%, or any amount of reduction in between as compared to native or control levels.

Ranges and values may be expressed herein as from “about” one particular value, and/or to “about” another particular value. When such a range is expressed, also specifically contemplated and considered disclosed is the range from the one particular value and/or to the other particular value unless the context specifically indicates otherwise. All of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed. As used herein, the term “about” and the like, when used in the context of a value, generally means plus or minus 10% of the value stated. For example, about 0.5 would include 0.45 and 0.55, about 10 would include 9 to 11, about 1000 would include 900 to 1100. It

By “treat” or “treatment” is meant a method of reducing the effects of a disease or condition. Treatment can also refer to a method of reducing the underlying cause of the disease or condition itself rather than just the symptoms. The treatment can be any reduction from native levels and/or any improvement of clinical signs of the disease and/or any increase in survival or function; and can be but is not limited to the complete ablation of the disease, condition, or the symptoms of the disease or condition. For example, a disclosed method for treating ARDS is considered to be a treatment if there is a reduction in one or more symptoms of the disease or if there is an improvement in the condition of the subject when compared to native levels in the same subject or control subjects. Thus, the reduction or improvement can be a 10, 20, 30, 40, 50, 60, 70, 80, 90, 100%, or any amount of reduction in between as compared to native or control levels. By “prevent” or “prevention” and the like is meant a method of preventing, or reducing the most severe complications of, a viral respiratory disease or disorder.

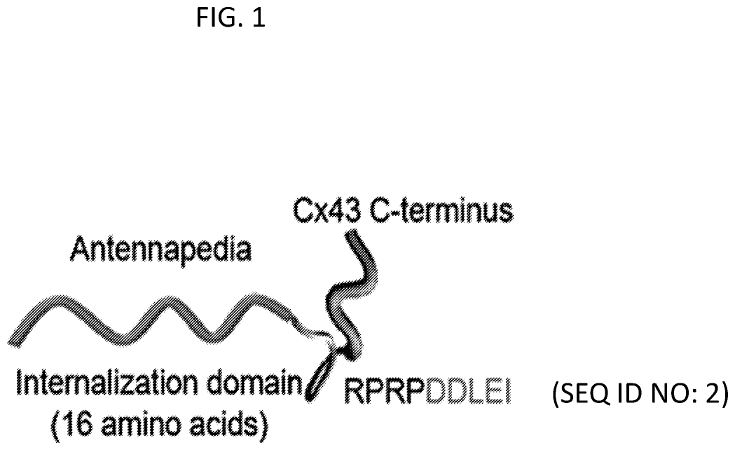

The present disclosure provides a novel method of use of a class of novel bioengineered Connexin43-based peptides that show therapeutic promise in the field of tissue engineering and regenerative medicine, including the injured lung epithelium⁸⁻¹⁰. The exemplary peptide aCT1 (FIG. 1) is a 25 aa peptide (3597.33 Da) that has a compact 2-domain design based on linkage of an Antennapedia cell internalization domain (1-16aa; RQPKIWFPNRRKPWKK; SEQ ID NO: 7) to the C-terminal PDZ binding domain of the transmembrane gap junction protein Cx43 (17-25aa; RPRPDDLEI; SEQ ID NO:2)¹⁰. Accordingly, the full aCT1 sequence is RQPKIWFPNRRKPWKK RPRPDDLEI (SEQ ID NO: 9). aCT1 and related peptides increase the size and stability of gap junctions by modulating the molecular interaction between Cx43 and its C-terminal binding partners, including the tight junction protein zonula occludens-1 (ZO-1). This leads to phosphorylation of the serine 368 (S368) amino acid on Cx43 and favors a transition of cell-surface Cx43 from hemichannels to gap junction intercellular channels. Phosphorylation of S368 prevents the binding of ZO-1 to the C-terminus of Cx43 long after aCT1 has degraded, permitting therapeutic longevity. Concomitantly, aCT1 stabilizes ZO-1 at the cell membrane, preventing junctional degradation in response to injury and preserving barrier function of epithelial cells¹¹. The result is stabilization of gap junctions (intercellular communication) as well as tight junctions (intercellular junctions) leading to a variety of beneficial effects including increased cellular communication, dampened inflammatory responses, and reduction in the infiltration and proliferation of profibrotic cells. Collectively, the molecular and cellular events facilitated by aCT1 preserves tissue integrity, reduces injury spread, dampens pathological inflammation, and accelerates healing and tissue regeneration.

aCT1's small, stable, soluble design facilitates direct translocation into cells without requirement for potentially toxic excipient compounds for intracellular drug delivery. aCT1 stabilizes intercellular junctions, reduces the release of proinflammatory cytokines, and promotes an effective epithelial response to injury.

aCT1 promotes the transition of Cx43 hemichannels to gap junctions, tempering inflammation. Cx43 hemichannels have critical roles in providing a paracrine route for cellular communication, regulating the release of molecules such as NAD+, ATP, glutamate, prostaglandin E2, and glutathione¹⁶⁻²⁶. The role of Cx43 hemichannels in the inflammatory response is particularly well defined, where hemichannel signaling regulates processes such as leukocyte chemotaxis, NO generation, and cytokine release^{24, 26-29}. The dysregulation of hemichannels that occurs in response to injury results in a pro-inflammatory state that prevents effective healing of damaged epithelial cells. Transition of cell-surface Cx43 from hemichannels to junctional intercellular channels with aCT1 treatment results in a coordinated reduction in the pro-inflammatory activity of hemichannels and stabilization of gap junctions, promoting intercellular communication. Furthermore, the action of aCT1 increases availability of the tight junction protein ZO-1 to interact with junctional proteins at the cell membrane. The resulting stabilization of intercellular junctions strengthens barrier integrity in pulmonary epithelial and endothelial cells¹¹.

Without wishing to be bound by theory, aCT1 directly targets and repairs the damaged cells lining airspaces and vasculature in the injured lung, thus restoring barrier integrity and directly addressing the cause of ARDS. While antiviral therapies in development for viruses such as SARS-CoV-2 can limit viral replication and accelerate viral clearance, these therapies will not address the need for rapid repair of the pulmonary epithelium in critically ill patients to prevent progression of severe pneumonia to ARDS.

Preclinical and clinical studies have demonstrated the safety and efficacy of the active pharmaceutical ingredient aCT1 peptide. Intravenous administration (bolus) of aCT1 in rats was well tolerated and a 5 mg/kg dosing level was established as the no-observable-adverse-effect level (NOAEL). Clinical signs were only observed at aCT1 administration levels that are well above the therapeutic range of efficacy, following intravenous administration of aCT1 at ≥10 mg/kg (Maximum Tolerated Dose). The results of these studies uniformly support aCT1 tolerability when delivered locally and systemically via various routes.

Clinical trials have been undertaken to assess safety of topical delivery of aCT1. These clinical trials have included over 474 human subjects with no drug related adverse events. These trials have demonstrated the efficacy and safety of local delivery of aCT1 to both acute and chronic skin injuries^{8, 30-32}. Furthermore, aCT1 was not immunogenic in any preclinical study or clinical trial (i.e. no anti-aCT1 antibodies were detected). The half-life of aCT1 in human blood is 15-20 mins (ex vivo studies) and pharmacokinetic studies included in clinical trials indicate no systemic exposure, underscoring local activity. Clinical trials to date have evaluated the safety, pharmacokinetics and immunogenicity of aCT1 when applied topically in maximal clinical use conditions, with favorable results.

The present disclosure provides experiments carried out to determine if an alpha Connexin polypeptide is useful in the treatment of COVID-19 patients to resolve symptoms of SARS-CoV-2 induced lung injury, thus reducing incidence and severity of ARDS. Surprisingly, the inventors of the present application found that the polypeptide aCT1, when administered to the lungs via intranasal or aerosolized delivery, is highly effective in treating, mitigating the symptoms of, and improving clinical outcomes associated with acute lung injury and ARDS.

The present disclosure is further illustrated by reference to the following Examples. However, it should be noted that these Examples, like the embodiments described above, are illustrative and are not to be construed as restricting the scope of the disclosure in any way.

### EXAMPLES

### Example 1. Efficacy of aCT1 Peptide in Treating Lung Injury

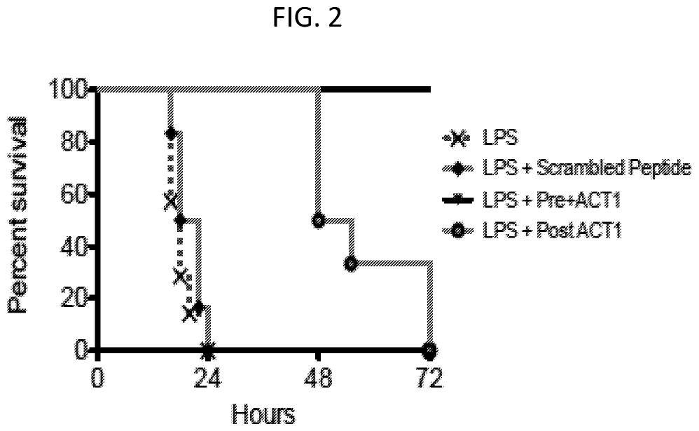

Studies were undertaken to assess whether delivery of aCT1 to the respiratory tract in animal models of ARDS would reduce epithelial and endothelial breakdown, thereby preserving the air-liquid barrier while preventing fluid accumulation and reducing immune cell infiltration, resulting in the amelioration of ARDS pathology and preserving lung function to improve survival. Using a well characterized mouse model of ARDS, we investigated whether aCT1 applied directly to the lungs could inhibit severe endotoxin induced lung injury. C57BL/6 mice were intranasally challenged with a lethal dose of lipopolysaccharide (LPS) and were treated with aCT1 immediately prior to LPS administration, or 6 h post LPS exposure (FIG. 2). Both aCT1 pre or post treatment significantly improved survival in response to a lethal dose of LPS.

### Example 2. Efficacy of aCT1 Peptide in a Model of Sepsis

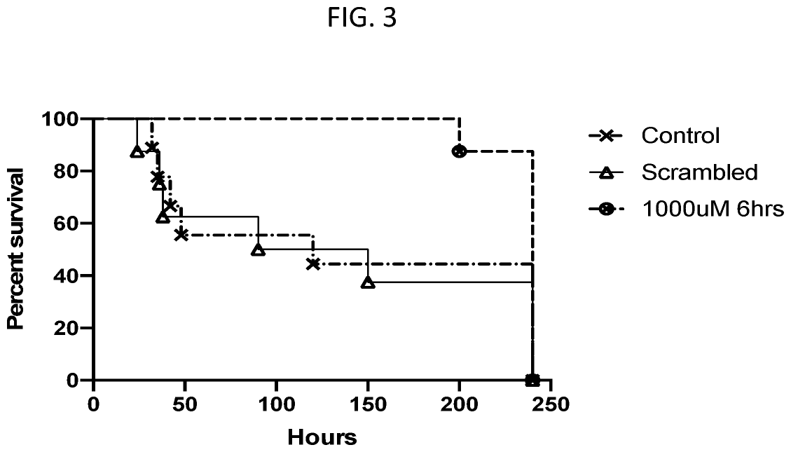

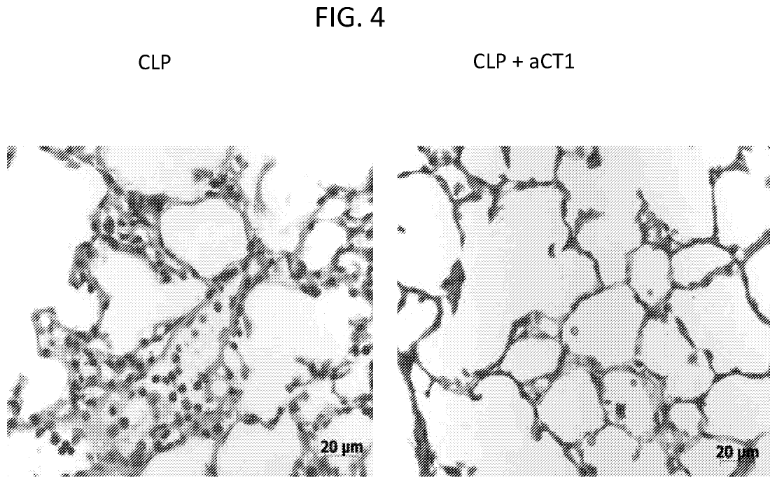

Sepsis is one of the most common causes of ARDS, causing diffuse inflammation in the lung and injury to the airway epithelium. In a well characterized cecal-ligation and puncture (CLP) mouse model of sepsis, treatment with aerosolized aCT1 prolonged survival (FIG. 3). Additionally, administration of aCT1 at 6 hrs post-CLP procedure significantly decreased immune cell infiltration and alveolar edema (FIG. 4).

### Example 3. Safety and Localization of aCT1 in Lung Tissue

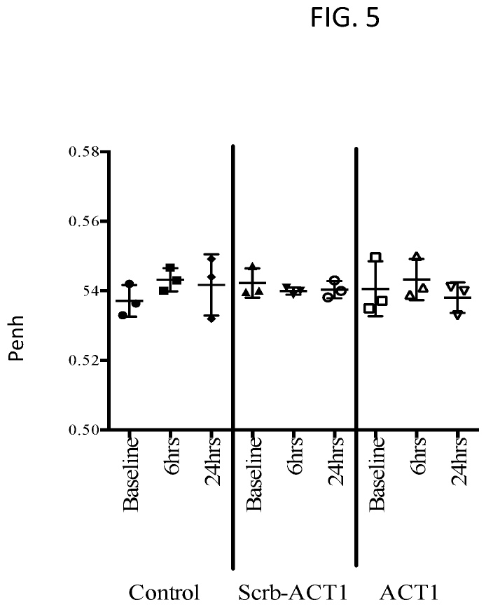

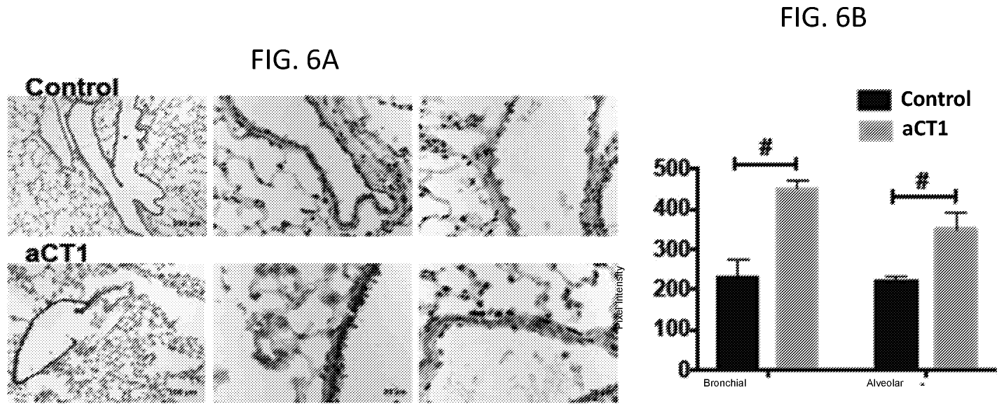

Aerosolized delivery of aCT1 to the lung is readily applicable in the hospital setting and would easily integrate into existing treatment paradigms for patients suffering from viral respiratory infections. To test the ability of aCT1 peptide to reach alveoli and airways, healthy mice were exposed to 1.5-5 mg/kg aerosolized aCT1. aCT1 was detected in bronchial and alveolar epithelial cells and the endothelial cells of the microvasculature. Critically, aerosolized administration of up to 5 mg/kg aCT1 had no effect on enhanced pause (Penh), a general measure of pulmonary function (FIG. 5). Delivery of up to 5 mg/kg aCT1 was not associated with alteration in lung function nor caused any visual signs of distress or labored breathing in healthy animals. FIG. 6A shows the localization of aCT1 staining 6 hours after nebulized delivery of aCT1 vs. saline control, in respiratory epithelial cells and endothelial cells of the microvasculature. FIG. 6B provides quantification of the aCT1 staining in bronchial and alveolar cells of animals that received aCT1 or saline control.

Taken together, the results of the studies indicate an alpha Connexin polypeptide (e.g., aCT1) prolongs survival in response to severe acute lung injuries, reduces inflammatory cell infiltration and edema, and has no adverse effect on pulmonary function in vivo. Treatment with such alpha Connexin polypeptides offers a unique therapeutic opportunity to modulate the lung injury response following viral respiratory infection by stabilizing intercellular junctions and tempering Cx43 hemichannel activity. Without wishing to be bound by theory, in the virally damaged lung, the alpha Connexin polypeptide will decrease lung inflammation, preserve the air-liquid barrier, and reduce injury spread. This surprisingly effective therapeutic benefit of aCT1 will translate in the clinic as a reduction in the severity of virus induced lung injury, promoting lung function and accelerating healing, thus preventing pneumonia progression and ARDS.

### Example 4. Evaluation of Therapeutic Efficacy of aCT1 Peptide in Preventing SARS-CoV-2 Induced Lung Injury

Animal Model of SARS-CoV-2: The MERS-CoV and SARS-CoV outbreaks of the 2000s highlighted the utility of non-human primates (NHPs) as the premiere translationally relevant species for modeling the human course of coronavirus diseases. The advancement and development of therapeutics to treat these severe respiratory infections in humans relied on NHPs; these animals are naturally susceptible to SARS and MERS-CoV infection and share many physiological similarities with humans. African green monkeys develop hallmarks of the severe lung injury induced by SARS- and MERS-CoVs, including clinically significant lesions to the alveolar and bronchiolar walls of the lung, inflammatory infiltration, and flooding of the airspaces leading to pulmonary edema^{33, 34}. Given the genetic and pathological similarities of SARS-CoV and SARS-CoV-2, the animal model of SARS-CoV in African green monkeys serves as a foundation for development of an animal model of SARS-CoV-2 in NHP^{4, 34, 35}. Accordingly, studies are undertaken to validate the efficacy and safety of aCT1 peptide as a therapeutic for lung injury, for example, SARS-CoV-2 induced lung injury.

A dose-ranging study using the African green monkey model of SARS-CoV-2 infection is designed to validate the safety and efficacy of aCT1 in a translationally relevant animal model and enable rapid progression to clinical evaluation in COVID-19 positive subjects. Monkeys are inoculated with SARS-CoV-2 and assigned to treatment groups. Efficacy of aCT1 is tested in a prophylactic treatment paradigm with administration at/01U the onset of infection (e.g., 1, 2, 3, 4, 5, 6, or 7 days post inoculation) and in a therapeutic treatment paradigm with administration beginning at the onset of symptoms (e.g., 7, 8, 9, 10, 11, 12, 13, 14, or more days post inoculation). A high dose and low dose of aCT1 are tested in each treatment paradigm. To confirm safety of lung delivery and complement aCT1′s existing toxicology package, a group of monkeys (male and female) receive daily aCT1 administered intranasally, without viral challenge. Exemplary treatment groups are provided in Table 3. Treatment is administered daily for the study duration. At 21 days post-inoculation, animals are euthanized and necropsied. Lung tissue is collected for quantitative analysis of viral RNA levels by qRT-PCR to confirm viral infection and quantify tissue burden.

TABLE 3
Exemplary Treatment Groups

| Group | Animal | Viral | | Treatment |
|---|---|---|---|---|
| Assignment | Number | Challenge | Treatment | Start |
| Group 1 | 4 (2M, 2F) | None | aCT1 (high | 7 day |
| | | | dose) | post inoc. |
| Group 2 | 8 (4 M, 4 F) | SARS-CoV-2 | Vehicle | 7 day |
| | | | | post inoc. |
| Group 3 | 8 (4 M, 4 F) | SARS-CoV-2 | aCT1 (high | 7 day |
| | | | dose) | post inoc. |
| Group 4 | 8 (4 M, 4 F) | SARS-CoV-2 | aCT1 (low | 7 day |
| | | | dose) | post inoc. |
| Group 5 | 8 (4 M, 4 F) | SARS-CoV-2 | aCT1 (High | 14 d |
| | | | dose) | post inoc. |
| Group 6 | 8 (4 M, 4 F) | SARS-CoV-2 | aCT1 (low | 14 d |
| | | | dose) | post inoc. |

Clinical Observations: Beginning on the day of inoculation (day 0), all animals are observed for signs of disease and clinical scores, including scoring of respiratory signs. Clinical examinations are performed on day 0, 7, and 14 with measurements of respiration rate of anesthetized animals.

Pulmonary Function: The therapeutic effect of aCT1 on pulmonary function is assessed using real time plethysmography to measure tidal volume and respiratory rate. These measurements and blood oxygenation (pulse oximetry) are performed on Day 21 prior to euthanasia. Demonstration of aCT1 efficacy in preserving lung function directly translates to decreased need for mechanical ventilation and improved survival.

Necropsy and Histopathology: Organs are examined grossly and findings are documented by a veterinary pathologist. Lung tissue samples are fixed, sectioned, and stained for histopathological scoring. Stained slides are analyzed and scored for inflammatory infiltrates, lung lesions, thickening of the alveolar septae, and alveolar edema by a veterinary pathologist. Improvement in lung histopathology scores with aCT1 treatment provide evidence of aCT1′s ability to prevent lung injury and inflammation, thus limiting severity of SARS-CoV-2 associated lung injury.

Safely Analyses: Adverse events are documented and designated as treatment related or non-treatment related by veterinary assessment. Adverse events are compared between all treatment groups. Data collected from non-viral challenged monkeys receiving daily intranasal aCT1 confirm a lack of any systemic effects of aCT1 as well as the safety of delivering aCT1 to the lung.

The results of the study show that aCT1 is effective in safe in preventing and treating lung injury and inflammation associated with viral infection.

### Example 5. aCT1 Efficacy in Lung Injury

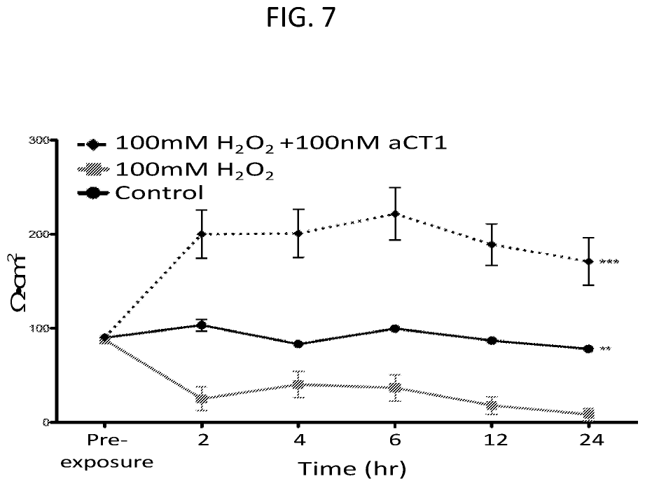

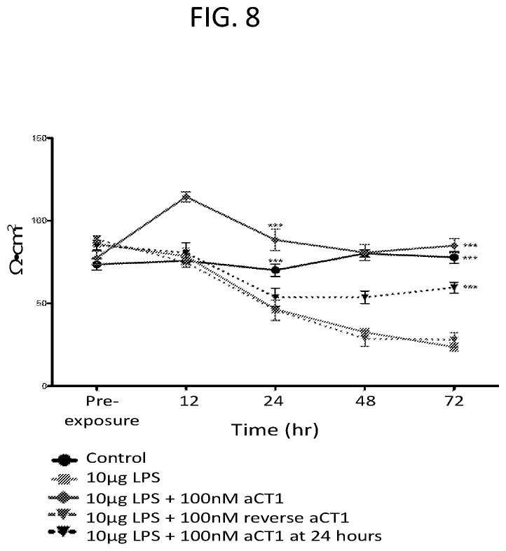

To determine if aCT1 treatment preserves junctional integrity in human lung cells, human bronchial epithelial cells (NHBEs) were grown as confluent monolayers on Transwell inserts and trans-epithelial resistance (TER) recorded. TER readings measure electrical resistance across a cell monolayer and provide a quantitative metric of intercellular junction integrity. Treatment with aCT1 stabilized intercellular junctions following exposure to 100 mM H₂O₂, demonstrating the ability of aCT1 to protect epithelial barrier integrity in response to oxidative stress in human lung cells (FIG. 7). aCT1 pretreatment of human lung microvascular endothelial cells (HLMEC) also stabilized endothelial barrier integrity in response to LPS endotoxin exposure. For additional clinical relevance, HLMECs were treated with aCT1 24 hr post LPS insult. aCT1 pre or post treatment preserved barrier integrity measured by TER, while untreated cells demonstrated decreased electrical conductivity indicative of junctional breakdown (FIG. 8). Taken together, the studies showed that aCT1 peptide stabilizes junctional barriers in human lung cells when administered pre and post insult.

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

### REFERENCES

1. Wu, C. et al. Risk Factors Associated With Acute Respiratory Distress Syndrome and Death in Patients With Coronavirus Disease 2019 Pneumonia in Wuhan, China. JAMA Intern Med (2020).
2. Koval, M. Claudin Heterogeneity and Control of Lung Tight Junctions. Annual Review of Physiology 75, 551-567 (2013).
3. Lin, L., Lu, L., Cao, W. & Li, T. Hypothesis for potential pathogenesis of SARS-CoV-2 infection—a review of immune changes in patients with viral pneumonia. Emerg Microbes Infect, 1-14 (2020).
4. Khan, S. et al. The emergence of a novel coronavirus (SARS-CoV-2), their biology and therapeutic options. J Clin Microbiol (2020).
5. Xu, Z. et al. Pathological findings of COVID-19 associated with acute respiratory distress syndrome. Lancet Respir Med (2020).
6. Yang, X. et al. Clinical course and outcomes of critically ill patients with SARS-CoV-2 pneumonia in Wuhan, China: a single-centered, retrospective, observational study. The Lancet Respiratory Medicine (2020).
7. Gu, J. & Korteweg, C. Pathology and pathogenesis of severe acute respiratory syndrome. Am J Pathol 170, 1136-1147 (2007).
8. Ghatnekar, G. S. et al. Connexin43 carboxyl-terminal peptides reduce scar progenitor and promote regenerative healing following skin wounding. Regen Med 4, 205-223 (2009).
9. Gourdie, R. G. et al. The unstoppable connexin43 carboxyl-terminus: new roles in gap junction organization and wound healing. Ann NY Acad Sci 1080, 49-62 (2006).
10. Rhett, J. M. et al. Novel therapies for scar reduction and regenerative healing of skin wounds. Trends Biotechnol 26, 173-180 (2008).
11. Obert, E. et al. Targeting the tight junction protein, zonula occludens-1, with the connexin43 mimetic peptide, aCT1, reduces VEGF-dependent RPE pathophysiology. J Mol Med (Berl) 95, 535-552 (2017).
12. Grek, C. L. et al. Topical administration of a connexin43-based peptide augments healing of chronic neuropathic diabetic foot ulcers: A multicenter, randomized trial. Wound Repair Regen 23, 203-212 (2015).
13. Ghatnekar, G., Grek, C., Armstrong, D., Desai, S. & Gourdie, R. The Effect of a Connexin43-based peptide on the Healing of Chronic Venous Leg Ulcers: A Multicenter, Randomized Trial. Journal of Investigative Dermatology (2014).
14. Grek, C. L. et al. A Multicenter Randomized Controlled Trial Evaluating a Cx43-Mimetic Peptide in Cutaneous Scarring. J Invest Dermatol 137, 620-630 (2017).
15. Ghatnekar, G. S., Grek, C. L., Armstrong, D. G., Desai, S. C. & Gourdie, R. G. The effect of a connexin43-based Peptide on the healing of chronic venous leg ulcers: a multicenter, randomized trial. The Journal of investigative dermatology 135, 289-298 (2015).
16. Rhett, J. M., Jourdan, J. & Gourdie, R. G. Connexin 43 connexon to gap junction transition is regulated by zonula occludens-1. Mol Biol Cell 22, 1516-1528 (2011).
17. Niessen, H., Harz, H., Bedner, P., Kramer, K. & Willecke, K. Selective permeability of different connexin channels to the second messenger inositol 1,4,5-trisphosphate. Journal of cell science 113 (Pt 8), 1365-1372 (2000).
18. Martin, P. & Parkhurst, S. M. Parallels between tissue repair and embryo morphogenesis. Development 131, 3021-3034 (2004).
19. Bruzzone, S., Guida, L., Zocchi, E., Franco, L. & De Flora, A. Connexin 43 hemi channels mediate Ca2+-regulated transmembrane NAD+fluxes in intact cells. FASEB J 15, 10-12 (2001).
20. Stout, C. E., Costantin, J. L., Naus, C. C. & Charles, A. C. Intercellular calcium signaling in astrocytes via ATP release through connexin hemichannels. J Biol Chem 277, 10482-10488 (2002).
21. Ye, Z. C., Wyeth, M. S., Baltan-Tekkok, S. & Ransom, B. R. Functional hemichannels in astrocytes: a novel mechanism of glutamate release. J Neurosci 23, 3588-3596 (2003).
22. Cherian, P. P. et al. Mechanical strain opens connexin 43 hemichannels in osteocytes: a novel mechanism for the release of prostaglandin. Mol Biol Cell 16, 3100-3106 (2005).
23. Rana, S. & Dringen, R. Gap junction hemichannel-mediated release of glutathione from cultured rat astrocytes. Neuroscience letters 415, 45-48 (2007).
24. Saez, J. C., Retamal, M. A., Basilio, D., Bukauskas, F. F. & Bennett, M. V. Connexin-based gap junction hemichannels: gating mechanisms. Biochimica et biophysica acta 1711, 215-224 (2005).
25. Evans, W. H. & Boitano, S. Connexin mimetic peptides: specific inhibitors of gap-junctional intercellular communication. Biochemical Society transactions 29, 606-612 (2001).
26. Ramachandran, S., Xie, L. H., John, S. A., Subramaniam, S. & Lal, R. A novel role for connexin hemichannel in oxidative stress and smoking-induced cell injury. PloS one 2, e712 (2007).
27. Rhett, J. M., Fann, S. A. & Yost, M. J. Purinergic Signaling in Early Inflammatory Events of the Foreign Body Response: Modulating Extracellular ATP as an Enabling Technology for Engineered Implants and Tissues. Tissue engineering. Part B, Reviews (2014).
28. Retamal, M. A., Cortes, C. J., Reuss, L., Bennett, M. V. & Saez, J. C. S-nitrosylation and permeation through connexin 43 hemichannels in astrocytes: induction by oxidant stress and reversal by reducing agents. Proceedings of the National Academy of Sciences of the United States of America 103, 4475-4480 (2006).
29. Retamal, M. A., Schalper, K. A., Shoji, K. F., Bennett, M. V. & Saez, J. C. Opening of connexin 43 hemichannels is increased by lowering intracellular redox potential. Proceedings of the National Academy of Sciences of the United States of America 104, 8322-8327 (2007).
30. Ghatnekar, G., Grek, C., Armstrong, D. G., Desai, S. C. & Gourdie, R. The Effect of a Connexin43-based peptide on the Healing of Chronic Venous Leg Ulcers: A Multicenter, Randomized Trial. J Invest Dermatol (2014).
31. Ghatnekar, G. S., Grek, C. L., Armstrong, D. G., Desai, S. C. & Gourdie, R. G. The effect of a connexin43-based Peptide on the healing of chronic venous leg ulcers: a multicenter, randomized trial. J Invest Dermatol 135, 289-298 (2015).
32. Grek, C. et al. A Multicenter, Randomized, Controlled Trial Evaluating a Connexin43-Mimetic Peptide in Cutaneous Scarring. Journal of Investigative Dermatology (2016).
33. McAuliffe, J. et al. Replication of SARS coronavirus administered into the respiratory tract of African Green, rhesus and cynomolgus monkeys. Virology 330, 8-15 (2004).
34. Clay, C. et al. Primary severe acute respiratory syndrome coronavirus infection limits replication but not lung inflammation upon homologous rechallenge. J Virol 86, 4234-4244 (2012).
35. Clay, C. C. et al. Severe acute respiratory syndrome-coronavirus infection in aged nonhuman primates is associated with modulated pulmonary and systemic immune responses. Immun Ageing 11, 4 (2014).

1. A method of treating or preventing a complication of a respiratory viral disease in a subject, comprising administering to the subject a composition comprising an isolated polypeptide comprising the carboxy terminal-most 4 to 30 contiguous amino acids of an alpha Connexin.

2. The method of claim 1, wherein the respiratory viral disease is caused by SARS-CoV-2.

3. The method of claim 1, wherein the respiratory viral disease is caused by an Influenza A, B, or C virus.

4. The method of claim 2, wherein the respiratory viral disease is SARS-CoV-2-induced ARDS and/or ALI.

5. The method of any one of claims 1-4, wherein the polypeptide comprises the amino acid sequence selected from the group consisting of SEQ ID NO: 1, SEQ ID NO: 2, SEQ ID NO: 3, SEQ ID NO: 4, and SEQ ID NO: 5.

6. The method of claim 5, wherein the polypeptide comprises the amino sequence of SEQ ID NO: 2.

7. The method of claim any one of claims 1-6, wherein the polypeptide further comprises a cellular internalization sequence.

8. The method of claim 7, wherein the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol).

9. The method of claim 8, wherein the cellular internalization sequence is Antennapedia, and wherein the sequence comprises the amino acid sequence of SEQ ID NO:7.

10. The method of claim any one of claims 1-9, wherein the polypeptide comprises the amino acid sequence selected from the group consisting of SEQ ID NO:8, SEQ ID NO:9, SEQ ID NO:10, SEQ ID NO:11, and SEQ ID NO:12.

11. The method of claim 10, wherein the polypeptide comprises the amino acid sequence of SEQ ID NO:9.

12. The method of any one of claims 1-11, wherein the polypeptide is administered to the subject parenterally, intranasally, intratracheally, by inhalant, or by topical intranasal administration.

13. The method of any one of claims 1-12, wherein the composition is administered to the subject by aerosolized delivery.

14. The method of any one of claims 1-13, wherein the composition is administered via an inhaler or a nebulizer.

15. The method of any one of claims 1-14, wherein the polypeptide is administered to the subject in a drug loaded microcarrier formulation.

16. The method of claim 15, wherein the drug loaded microcarrier formulation comprises nanoparticles or exosomes.

17. The method of any one of claims 1-16, wherein the polypeptide is administered to the subject at the onset of infection.

18. The method of any one of claims 1-16, wherein the polypeptide is administered to the subject prior to the onset of symptoms of the respiratory viral disease.

19. The method of any one of claim s1-16, wherein the polypeptide is administered to the subject after onset of symptoms of the respiratory viral disease.

20. The method of any one of claims 1-19, wherein the method prevents lung injury caused by the respiratory viral disease.

21. The method of any one of claims 1-20, wherein the method limits the progression of lung injury caused by the respiratory viral disease.

22. The method of claim any one of claims 1-21, wherein the method maintains lung function after the onset of the respiratory viral disease.

23. A method for treating or preventing a respiratory disease or disorder in a subject in need thereof, the method comprising administering to the subject a composition comprising an isolated polypeptide comprising the carboxy terminal-most 4 to 30 contiguous amino acids of an alpha Connexin.

24. The method of claim 23, wherein the respiratory disease or disorder is acute respiratory distress syndrome (ARDS), alcoholic lung syndrome, acute lung injury, chronic obstructive pulmonary disease (COPD), or pulmonary fibrosis.

25. The method of claim 24, wherein the respiratory disease or disorder is idiopathic pulmonary fibrosis (IPF).

26. The method of any one of claims 23-25, wherein the polypeptide comprises the amino acid sequence selected from the group consisting of SEQ ID NO: 1, SEQ ID NO: 2, SEQ ID NO: 3, SEQ ID NO: 4, and SEQ ID NO: 5.

27. The method of claim 26, wherein the polypeptide comprises the amino sequence of SEQ ID NO: 2.

28. The method of claim any one of claims 23-28, wherein the polypeptide further comprises a cellular internalization sequence.

29. The method of claim 28, wherein the cellular internalization sequence comprises an amino acid sequence of a protein selected from a group consisting of Antennapedia, TAT, HIV-Tat, Penetratin, Antp-3A (Antp mutant), Buforin II, Transportan, MAP (model amphipathic peptide), K-FGF, Ku70, Prion, pVEC, Pep-1, SynB 1, Pep-7, HN-1, BGSC (Bis-Guanidinium-Spermidine-Cholesterol) and BGTC (Bis-Guanidinium-Tren-Cholesterol).

30. The method of claim 29, wherein the cellular internalization sequence is Antennapedia, and wherein the sequence comprises the amino acid sequence of SEQ ID NO:7.

31. The method of claim any one of claims 23-30, wherein the polypeptide comprises the amino acid sequence selected from the group consisting of SEQ ID NO:8, SEQ ID NO:9, SEQ ID NO:10, SEQ ID NO:11, and SEQ ID NO:12.

32. The method of claim 31, wherein the polypeptide comprises the amino acid sequence of SEQ ID NO:9.

33. The method of any one of claims 23-32, wherein the polypeptide is administered to the subject parenterally, intranasally, intratracheally, by inhalant, or by topical intranasal administration.

34. The method of any one of claims 23-33, wherein the composition is administered to the subject by aerosolized delivery.

35. The method of any one of claims 24-34, wherein the composition is administered via an inhaler or a nebulizer.

36. The method of any one of claims 23-35, wherein the polypeptide is administered to the subject in a drug loaded microcarrier formulation.

37. The method of claim 36, wherein the drug loaded microcarrier formulation comprises nanoparticles or exosomes.

38. A composition for use in treating or preventing a complication of a respiratory viral disease in a subject, wherein the composition comprises a polypeptide comprising the carboxy terminal-most 4 to 30 contiguous amino acids of an alpha Connexin.

39. A composition for use in treating or preventing a respiratory disorder in a subject, wherein the composition comprises a polypeptide comprising the carboxy terminal-most 4 to 40 contiguous amino acids of an alpha Connexin.


SEQUENCE LISTING (91 sequences)

SEQ ID NO: 1 - protein, 20 aa, Artificial Sequence
  Description of Artificial Sequences; note = synthetic construct
PSSRASSRASSRPRPDDLEI

SEQ ID NO: 2 - protein, 9 aa, Artificial Sequence
  Description of Artificial Sequences; note = synthetic construct
RPRPDDLEI

SEQ ID NO: 3 - protein, 9 aa, Artificial Sequence
  Description of Artificial Sequences; note = synthetic construct
RPRPDDLEV

SEQ ID NO: 4 - protein, 9 aa, Artificial Sequence
  Description of Artificial Sequences; note = synthetic construct
RPRPDDVPV

SEQ ID NO: 5 - protein, 9 aa, Artificial Sequence
  Description of Artificial Sequences; note = synthetic construct
KARSDDLSV

SEQ ID NO: 6 - DNA, 27 bp, Artificial Sequence
  Description of Artificial Sequences; note = synthetic construct
agacctcggcctgatgacctggagatt

SEQ ID NO: 7 - protein, 16 aa, Artificial Sequence
  Description of Artificial Sequences; note = synthetic construct
RQPKIWFPNRRKPWKK

SEQ ID NO: 8 - protein, 36 aa, Artificial Sequence
  Description of Artificial Sequences; note = synthetic construct
RQPKIWFPNRRKPWKKPSSRASSRASSRPRPDDLEI

SEQ ID NO: 9 - protein, 25 aa, Artificial Sequence
  Description of Artificial Sequences; note = synthetic construct
RQPKIWFPNRRKPWKKRPRPDDLEI

SEQ ID NO: 10 - protein, 25 aa, Artificial Sequence
  Description of Artificial Sequences; note = synthetic construct
RQPKIWFPNRRKPWKKRPRPDDLEV

SEQ ID NO: 11 - protein, 25 aa, Artificial Sequence
  Description of Artificial Sequences; note = synthetic construct
RQPKIWFPNRRKPWKKRPRPDDVPV

SEQ ID NO: 12 - protein, 25 aa, Artificial Sequence
  Description of Artificial Sequences; note = synthetic construct
RQPKIWFPNRRKPWKKKARSDDLSV

SEQ ID NO: 13 - DNA, 74 bp, Artificial Sequence
  Description of Artificial Sequences; note = synthetic construct
cggcagcccaagatctggttccccaaccggcggaagccctggaagaagcggcccggcccgacgacctggagatc

SEQ ID NO: 14 - protein, 11 aa, Artificial Sequence
  Description of Artificial Sequences; note = synthetic construct
GRKKRRQRPPQ

SEQ ID NO: 15 - protein, 16 aa, Artificial Sequence
  Description of Artificial Sequences; note = synthetic construct
RQIKIWFQNRRMKWKK

SEQ ID NO: 16 - protein, 16 aa, Artificial Sequence
  Description of Artificial Sequences; note = synthetic construct
RQIAIWFQNRRMKWAA

SEQ ID NO: 17 - protein, 9 aa, Artificial Sequence
  Description of Artificial Sequences; note = synthetic construct
RKKRRQRRR

SEQ ID NO: 18 - protein, 21 aa, Artificial Sequence
  Description of Artificial Sequences; note = synthetic construct
TRSSRAGLQFPVGRVHRLLRK

SEQ ID NO: 19 - protein, 26 aa, Artificial Sequence
  Description of Artificial Sequences; note = synthetic construct
GWTLNSAGYLLGKINKALAALAKKIL

SEQ ID NO: 20 - protein, 18 aa, Artificial Sequence
  Description of Artificial Sequences; note = synthetic construct
KLALKLALKALKAALKLA

SEQ ID NO: 21 - protein, 16 aa, Artificial Sequence
  Description of Artificial Sequences; note = synthetic construct
AAVALLPAVLLALLAP

SEQ ID NO: 22 - protein, 10 aa, Artificial Sequence
  Description of Artificial Sequences; note = synthetic construct
VPMLKPMLKE

SEQ ID NO: 23 - protein, 28 aa, Artificial Sequence
  Description of Artificial Sequences; note = synthetic construct
MANLGYWLLALFVTMWTDVGLCKKRPKP

SEQ ID NO: 24 - protein, 18 aa, Artificial Sequence
  Description of Artificial Sequences; note = synthetic construct
LLIILRRRIRKQAHAHSK

SEQ ID NO: 25 - protein, 21 aa, Artificial Sequence
  Description of Artificial Sequences; note = synthetic construct
KETWWETWWTEWSQPKKKRKV

SEQ ID NO: 26 - protein, 18 aa, Artificial Sequence
  Description of Artificial Sequences; note = synthetic construct
RGGRLSYSRRRFSTSTGR

SEQ ID NO: 27 - protein, 15 aa, Artificial Sequence
  Description of Artificial Sequences; note = synthetic construct
SDLWEMMMVSLACQY

SEQ ID NO: 28 - protein, 12 aa, Artificial Sequence
  Description of Artificial Sequences; note = synthetic construct
TSPLNIHNGQKL

SEQ ID NO: 29 - protein, 19 aa, Artificial Sequence
  Description of Artificial Sequences; note = synthetic construct
PSRASSRASSRPRPDDLEI

SEQ ID NO: 30 - protein, 20 aa, Artificial Sequence
  Description of Artificial Sequences; note = synthetic construct
GSNKSTASSKSPDPKNSVWI

SEQ ID NO: 31 - protein, 20 aa, Artificial Sequence
  Description of Artificial Sequences; note = synthetic construct
GSNKSSASSKSGDGKNSVWI

SEQ ID NO: 32 - protein, 21 aa, Artificial Sequence
  Description of Artificial Sequences; note = synthetic construct
GRASKASRASSGRARPEDLAI

SEQ ID NO: 33 - protein, 13 aa, Artificial Sequence
  Description of Artificial Sequences; note = synthetic construct
GSASSRDGKTVWI

SEQ ID NO: 34 - protein, 19 aa, Artificial Sequence
  Description of Artificial Sequences; note = synthetic construct
PRVSVPNFGRTQSSDSAYV

SEQ ID NO: 35 - protein, 19 aa, Artificial Sequence
  Description of Artificial Sequences; note = synthetic construct
PRMSMPNFGRTQSSDSAYV

SEQ ID NO: 36 - protein, 21 aa, Artificial Sequence
  Description of Artificial Sequences; note = synthetic construct
PRAGSEKGSASSRDGKTTVWI

SEQ ID NO: 37 - protein, 23 aa, Artificial Sequence
  Description of Artificial Sequences; note = synthetic construct
GYHSDKRRLSKASSKARSDDLSV

SEQ ID NO: 38 - protein, 19 aa, Artificial Sequence
  Description of Artificial Sequences; note = synthetic construct
PLSRLSKASSRARSDDLTV

SEQ ID NO: 39 - protein, 22 aa, Artificial Sequence
  Description of Artificial Sequences; note = synthetic construct
PNHVVSLTNNLIGRRVPTDLQI

SEQ ID NO: 40 - protein, 29 aa, Artificial Sequence
  Description of Artificial Sequences; note = synthetic construct
PSCVSSSAVLTTICSSDQVVPVGLSSFYM

SEQ ID NO: 41 - protein, 21 aa, Artificial Sequence
  Description of Artificial Sequences; note = synthetic construct
GRSSKASKSSGGRARAADLAI

SEQ ID NO: 42 - protein, 17 aa, Artificial Sequence
  Description of Artificial Sequences; note = synthetic construct
LCYLLIRYCSGKSKKPV

SEQ ID NO: 43 - protein, 18 aa, Artificial Sequence
  Description of Artificial Sequences; note = synthetic construct
GQKPPSRPSSSASKKQYV

SEQ ID NO: 44 - protein, 19 aa, Artificial Sequence
  Description of Artificial Sequences; note = synthetic construct
SSRASSRASSRPRPDDLEV

SEQ ID NO: 45 - protein, 9 aa, Artificial Sequence
  Description of Artificial Sequences; note = synthetic construct
RPKPDDLEI

SEQ ID NO: 46 - protein, 19 aa, Artificial Sequence
  Description of Artificial Sequences; note = synthetic construct
SSRASSRASSRPKPDDLEI

SEQ ID NO: 47 - protein, 9 aa, Artificial Sequence
  Description of Artificial Sequences; note = synthetic construct
RPKPDDLDI

SEQ ID NO: 48 - protein, 19 aa, Artificial Sequence
  Description of Artificial Sequences; note = synthetic construct
SSRASSRASSRPRPDDLDI

SEQ ID NO: 49 - protein, 19 aa, Artificial Sequence
  Description of Artificial Sequences; note = synthetic construct
SSRASTRASSRPRPDDLEI

SEQ ID NO: 50 - protein, 9 aa, Artificial Sequence
  Description of Artificial Sequences; note = synthetic construct
RPRPEDLEI

SEQ ID NO: 51 - protein, 19 aa, Artificial Sequence
  Description of Artificial Sequences; note = synthetic construct
SSRASSRASSRPRPEDLEI

SEQ ID NO: 52 - protein, 9 aa, Artificial Sequence
  Description of Artificial Sequences; note = synthetic construct
GDGKNSVWV

SEQ ID NO: 53 - protein, 23 aa, Artificial Sequence
  Description of Artificial Sequences; note = synthetic construct
SKAGSNKSTASSKSGDGKNSVWV

SEQ ID NO: 54 - protein, 18 aa, Artificial Sequence
  Description of Artificial Sequences; note = synthetic construct
GQKPPSRPSSSASKKLYV

SEQ ID NO: 55 - protein, 24 aa, Artificial Sequence
  Description of Artificial Sequences; note = synthetic construct
RQPKIWFPNRRKPWKIELDDPRPR

SEQ ID NO: 56 - protein, 20 aa, Artificial Sequence
  Description of Artificial Sequences; note = synthetic construct
GRKKRRQRPPQRPRPDDLEI

SEQ ID NO: 57 - protein, 25 aa, Artificial Sequence
  Description of Artificial Sequences; note = synthetic construct
RQIKIWFQNRRMKWKKRPRPDDLEI

SEQ ID NO: 58 - protein, 25 aa, Artificial Sequence
  Description of Artificial Sequences; note = synthetic construct
RQIAIWFQNRRMKWAARPRPDDLEI

SEQ ID NO: 59 - protein, 18 aa, Artificial Sequence
  Description of Artificial Sequences; note = synthetic construct
RKKRRQRRRRPRPDDLEI

SEQ ID NO: 60 - protein, 30 aa, Artificial Sequence
  Description of Artificial Sequences; note = synthetic construct
TRSSRAGLQFPVGRVHRLLRKRPRPDDLEI

SEQ ID NO: 61 - protein, 35 aa, Artificial Sequence
  Description of Artificial Sequences; note = synthetic construct
GWTLNSAGYLLGKINKALAALAKKILRPRPDDLEI

SEQ ID NO: 62 - protein, 27 aa, Artificial Sequence
  Description of Artificial Sequences; note = synthetic construct
KLALKLALKALKAALKLARPRPDDLEI

SEQ ID NO: 63 - protein, 25 aa, Artificial Sequence
  Description of Artificial Sequences; note = synthetic construct
AAVALLPAVLLALLAPRPRPDDLEI

SEQ ID NO: 64 - protein, 19 aa, Artificial Sequence
  Description of Artificial Sequences; note = synthetic construct
VPMLKPMLKERPRPDDLEI

SEQ ID NO: 65 - protein, 37 aa, Artificial Sequence
  Description of Artificial Sequences; note = synthetic construct
MANLGYWLLALFVTMWTDVGLCKKRPKPRPRPDDLEI

SEQ ID NO: 66 - protein, 27 aa, Artificial Sequence
  Description of Artificial Sequences; note = synthetic construct
LLIILRRRIRKQAHAHSKRPRPDDLEI

SEQ ID NO: 67 - protein, 30 aa, Artificial Sequence
  Description of Artificial Sequences; note = synthetic construct
KETWWETWWTEWSQPKKKRKVRPRPDDLEI

SEQ ID NO: 68 - protein, 27 aa, Artificial Sequence
  Description of Artificial Sequences; note = synthetic construct
RGGRLSYSRRRFSTSTGRRPRPDDLEI

SEQ ID NO: 69 - protein, 24 aa, Artificial Sequence
  Description of Artificial Sequences; note = synthetic construct
SDLWEMMMVSLACQYRPRPDDLEI

SEQ ID NO: 70 - protein, 21 aa, Artificial Sequence
  Description of Artificial Sequences; note = synthetic construct
TSPLNIHNGQKLRPRPDDLEI

SEQ ID NO: 71 - protein, 120 aa, Artificial Sequence
  Description of Artificial Sequences; note = synthetic construct
KGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVD

SEQ ID NO: 72 - protein, 120 aa, Artificial Sequence
  Description of Artificial Sequences; note = synthetic construct
KTDPYSHSGTMSPSKDCGSPKYAYYNGCSSPTAPLSPMSPPGYKLVTGDRNNSSCRNYNKQASEQNWANYSAEQNRMGQAGSTISNSHAQPFDFADEHQNTKKLASGHELQPLTIVDQRP

SEQ ID NO: 73 - protein, 120 aa, Artificial Sequence
  Description of Artificial Sequences; note = synthetic construct
LGFGTIRDSLNSKRRELEDPGAYNYPFTWNTPSAPPGYNIAVKPDQIQYTELSNAKIAYKQNKANTAQEQQYGSHEENLPADLEALQREIRMAQERLDLAVQAYSHQNNPHGPREKKAKV

SEQ ID NO: 74 - protein, 120 aa, Artificial Sequence
  Description of Artificial Sequences; note = synthetic construct
GFGTIRDTLNNKRKELEDSGTYNYPFTWNTPSAPPGYNIAVKPDQMQYTELSNAKMAYKQNKANIAQEQQYGSNEENIPADLENLQREIKVAQERLDMAIQAYNNQNNPGSSSREKKSKA

SEQ ID NO: 75 - protein, 120 aa, Artificial Sequence
  Description of Artificial Sequences; note = synthetic construct
PYLVDCFVSRPTEKTIFIIFMLVVGLISLVLNLLELVHLLCRCLSRGMRARQGQDAPPTQGTSSDPYTDQVFFYLPVGQGPSSPPCPTYNGLSSSEQNWANLTTEERLASSRPPLFLDPP

SEQ ID NO: 76 - protein, 120 aa, Artificial Sequence
  Description of Artificial Sequences; note = synthetic construct
CGSKEHGNRKMRGRLLLTYMASIFFKSVFEVAFLLIQWYLYGFTLSAVYICEQSPCPHRVDCFLSRPTEKTIFILFMLVVSMVSFVLNVIELFYVLFKAIKNHLGNEKEEVYCNPVELQK

SEQ ID NO: 77 - protein, 239 aa, Artificial Sequence
  Description of Artificial Sequences; note = synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 78 - DNA, 60 bp, Artificial Sequence
  Description of Artificial Sequences; note = synthetic construct
ccctcctcccgggcctcctcccgggcctcctcccggccccggcccgacgacctggagatc

SEQ ID NO: 79 - DNA, 27 bp, Artificial Sequence
  Description of Artificial Sequences; note = synthetic construct
cggccccggcccgacgacctggagatc

SEQ ID NO: 80 - DNA, 27 bp, Artificial Sequence
  Description of Artificial Sequences; note = synthetic construct
cggccccggcccgacgacctggaggtg

SEQ ID NO: 81 - DNA, 27 bp, Artificial Sequence
  Description of Artificial Sequences; note = synthetic construct
cggccccggcccgacgacgtgcccgtg

SEQ ID NO: 82 - DNA, 27 bp, Artificial Sequence
  Description of Artificial Sequences; note = synthetic construct
aaggcccggtccgacgacctgtccgtg

SEQ ID NO: 83 - DNA, 48 bp, Artificial Sequence
  Description of Artificial Sequences; note = synthetic construct
cggcagcccaagatctggttccccaaccggcggaagccctggaagaag

SEQ ID NO: 84 - DNA, 108 bp, Artificial Sequence
  Description of Artificial Sequences; note = synthetic construct
cggcagcccaagatctggttccccaaccggcggaagccctggaagaagccctcctcccgggcctcctcccgggcctcctcccggccccggcccgacgacctggagatc

SEQ ID NO: 85 - DNA, 75 bp, Artificial Sequence
  Description of Artificial Sequences; note = synthetic construct
cggcagcccaagatctggttccccaaccggcggaagccctggaagaagcggccccggcccgacgacctggagatc

SEQ ID NO: 86 - DNA, 75 bp, Artificial Sequence
  Description of Artificial Sequences; note = synthetic construct
cggcagcccaagatctggttccccaaccggcggaagccctggaagaagcggccccggcccgacgacctggaggtg

SEQ ID NO: 87 - DNA, 75 bp, Artificial Sequence
  Description of Artificial Sequences; note = synthetic construct
cggcagcccaagatctggttccccaaccggcggaagccctggaagaagcggccccggcccgacgacgtgcccgtg

SEQ ID NO: 88 - DNA, 75 bp, Artificial Sequence
  Description of Artificial Sequences; note = synthetic construct
cggcagcccaagatctggttccccaaccggcggaagccctggaagaagaaggcccggtccgacgacctgtccgtg

SEQ ID NO: 89 - protein, 20 aa, Artificial Sequence
  Description of Artificial Sequences; note = synthetic construct
PCSRASSRMSSRARPDDLDV

SEQ ID NO: 90 - protein, 19 aa, Artificial Sequence
  Description of Artificial Sequences; note = synthetic construct
PRVSVPNFGRTQSSDSAYV

SEQ ID NO: 91 - protein, 26 aa, Artificial Sequence
  Description of Artificial Sequences; note = synthetic construct
  misc_feature (1)..(1): Xaa is biotinylated
  misc_feature (1)..(1): Xaa is L-2-aminohexanoic acid
XRQPKIWFPNRRKPWKKRPRPDDLEI